(19) United States
(12) Patent Application Publication
(10) Pub. No.: US 2007/0022960 A1
(43) Pub. Date: Feb. 1, 2007

(54) Method for the production of an agent from larvae in flies for treating wounds and agent produced according to said method

(76) Inventor: Wihelm Fleischmann, Bietigheim-Bissingen (DE)

Correspondence Address: AKERMAN SENTERFITT, P.O. BOX 3188, WEST PALM BEACH, FL, 33402-3188, US

(21) Appl. No.: 10/571,218

(22) PCT Filed: Aug. 18, 2004
(86) PCT No.: PCT/EP04/09244

§ 371 Date: Sep. 1, 2006

(30) Foreign Application Priority Data
Sep. 10, 2003 (DE) 103 42 104.1

(51) Int. Cl. A01K 67/02

(52) U.S. Cl. 119/6.6

(57) ABSTRACT

The treatment of wounds by means of larvae from flies is improved by virtue of the fact that the larvae are contaminated with bacteriophages.

### CROSS REFERENCE TO RELATED APPLICATION

This application is a national stage of PCT/EP2004/009244 filed Aug. 18, 2004 and based upon DE 103 42 104.1 filed Sep. 10, 2003 under the International Convention.

### BACKGROUND OF THE INVENTION

1. Field of the invention

The invention concerns a process for the production of a means for treating a wound.

2. Description of Related Art

It is known that the larvae of flies, in particular the larvae of flies of genus Lucilia, have an effect of promoting healing of wounds. The secretion secreted by the living larvae stimulates tissue proliferation, liquefies necrotic tissue and scabs by proteolysis, and has an antiseptic effect against certain bacteria types, for example Streptococcus and Staphylococcus. The use of living larvae for treatment of wounds is described in, for example, DE 19901134A1.

Further, to simplify the production and application of larvae in that the eggs of the flies, it is known from DE 19925996A1 to bring the larvae in an early stage of development into environmental conditions which interrupt the development cycle. The breeding or rearing of larvae can in this manner be carried out in a sterile manner in a commercial undertaking. The eggs or the larvae are brought, at an early development stage, prior to onset of their active phase, into the development-inhibiting environment and are packaged in this development-inhibiting environment. The eggs, or as the case may be, the larvae, can be transported in this condition from one location to another and be stored by the manufacturer or the end user in this condition for prolonged period of time. Prior to the therapeutic application, the eggs or, as the case may be, the larvae, are then brought into an environment in which the development cycle can resume, so that the larvae enter into their active phase, in which they can be applied to the wound.

### SUMMARY OF THE INVENTION

The invention is concerned with the task of improving the effectiveness of wound treatment with larvae.

This task is inventively solved by a process for producing a means for wound treatment.

Advantageous embodiments of the invention are set forth in the dependent subclaims.

The essential concept of the invention is comprised therein, that the larvae therapy is to be enhanced by an application of bacteriophages.

Bacteriophages, also referred to as phages, are viruses, of which the host cells are bacteria. The phages penetrate into the bacteria and replicate in the inside of the bacteria cells. Temperate phages carry out a lysogenic condition in the bacteria, in which the bacteria can survive. Virulent or lytic phages multiply very rapidly in the bacteria and disrupt this, so that they are released in the subsequent lysis. It is known to employ living phages for treatment of bacterial infections. Therein it is necessary to employ phages with the highest possible virulence against the target bacteria. These phages are particularly suited for treatment of infections, since in comparison to frequently employed broad-spectrum antibiotics they have hardly any side effects due to their high specificity. In particular, the phages can also kill germs that exhibit a multi-resistance against antibiotics. Therein it is advantageous that the phages multiply very rapidly exponentially until their nutrient reserve is exhausted, that is, until bacteria no longer are present. In the manner typical for virus they can now transition into the lifeless rest condition (virions), in which they remain until a renewed contact with a specific target bacteria again revives their reproduction.

### DETAILED DESCRIPTION OF THE INVENTION

The invention is based upon a synergistic interaction between the larvae and the bacteriophages.

The larvae are mobile and can, in accordance with the invention, serve as transport means and carriers for the phages, thereby bringing the immobile phages into the areas of the wound to be treated. Therein it is important that the larvae become active in the wound areas in which necrotized tissue is located and thus where an increased danger of infection exists. By means of the larvae thus a local targeted application of phages is made possible. Therein the contamination of the larvae with the phages supplementally has the advantage, that the phages are not washed away by the wound discharge or emissions, which thus improves their effectiveness.

The phages can not remove or penetrate bandages or other barriers, which surround necrotic and infected tissue. There is thus the advantage here in accordance with the invention that the larvae with their sharp teeth and horns and their necrolytic secretion can attack and penetrate such wound coverings and barriers, so that the phages can become effective with high efficiency.

The phages require for their replication and effectiveness an environmental temperature maintained as precisely as possible, for example at 37° C. and preferably a alkali environment. Here also there is in accordance with the invention an advantageous interaction, in which the larvae produce an alkali wound environment with their secretion and by their metabolism cause a temperature rise at the wound surface. Thereby the larvae produce optimal environment conditions for the replication of the phages. The antiseptic effect of the larval secretion is limited to specific causative organisms or pathogens, in particular to Streptococci and Staphylococci. In the course of prolonged larval treatment, thus, by the alkali wound environment, advantageously a super-infection of the wound with grahm-negative pathogens such as Pseudomona and Proteus can be fostered. These problems of a pure larval therapy can be avoided, or at least strongly be minimized, by the contamination of the larvae with selected pathogens. The antiseptic and antibiotic spectrum of action of the larval secretion is strongly advanced by the supplemental application of the phages.

In order to achieve an optimal prophylaxis and therapy of wound infections, it is useful to make available a broad spectrum of specific phages from which to select, which target the various bacteria to be combated. Thereby it is also possible, to reduce the likelihood of development of resistance to the phages.

For a targeted application of the phages, it is important to breed the flies in a sterile-as-possible environment, so that the eggs of the flies are preferably already obtained in a substantially germ-free environment. The breeding of the larvae from the egg stage occurs under sterile conditions. The contamination with the selected strains of phages could occur already in the egg stage or also in the subsequent larval stage. Since the phages remain in their lifeless virion rest condition so long as no target bacteria are present, the rearing of the larvae is possible even in the case of an early contamination, for example in the egg stage.

Preferably at a very early development stage in the production process the eggs of the flies or the larvae are introduced into an environmental condition in which the development cycle is stopped. Such an environmental condition could be for example a reduced temperature, a vacuum or an inert atmosphere, or as a consequence of removal of moisture. In this condition the eggs or larvae contaminated with the bacteriophages can be preserved in the manufacturing facility and be transported to the end user. After reaching the end user a further storage is also possible, until the larvae are applied for wound treatment. Prior to the planned application the eggs or, as the case may be, the larvae in the early development stage, are brought out of the development inhibiting environmental condition into an environment in which the development cycle of the larvae can proceed, so that the larvae can rapidly develop into their active phase, in which condition they are applied to the wound. In the storage and the transport the phages can supplementally also ensure the maintenance of the germ-free state of the larval product, which is necessary for a therapeutic application.

The synergistic effect of the larval secretion and the bacteriophages can also be taken advantage of in the manner, that the larval secretion and the phages are applied jointly upon a wound inlay or padding or intermediate layer, which is then applied upon the wound surface. The larval secretion can be secreted from the living larvae onto the wound insert, can be obtained by rinsing off of the larvae, or can be extracted from the larvae. The necrotic effect of the larval secretion, the decomposition of barriers which block the effect of the phages, and the alkali influence of the wound environment also provides support here.

1. A process for producing a means for wound treatment, in which larvae to be applied to the wound to be treated are raised from the eggs of flies, wherein the larvae are contaminated with bacteriophages in the manner that the larvae to be applied become the carriers of the bacteriophages.

2. A process according to claim 1, wherein at least the rearing of the larvae from the egg stage is carried out under sterile or germ suppressing environmental conditions.

3. A process according to claim 1 or 2, wherein the contamination with the bacteriophages is carried out in the egg stage.

4. A process according to claim 1, wherein the contamination with the bacteriophages is carried out in the development stage of the larvae, before the larvae become active for wound treatment.

5. A process according to claim 1, wherein the eggs or the larvae are contaminated with bacteriophages in the early larval stage, and that the eggs or, as the case may be, the larvae in their early development stage are brought into an environmental condition for storage and/or transport in which the development cycle is arrested.

6. A composition for wound treatment, which contains living larvae of flies, which larvae are to be applied to the wound to be treated, wherein the larvae are contaminated with bacteriophages.

7. A composition according to claim 6, wherein the larvae prior to contamination with the bacteriophages are germ-free.

8. A composition according to claim 6, wherein the larvae are contaminated with the bacteriophages in the egg stage or, as the case may be, an early larval stage, and enclosed in an environment in which their development cycle is inhibited.

9. A process for wound treatment with larvae of flies, in which living larvae are applied to the wound, wherein the larvae are contaminated with bacteriophages and carry these bacteriophages into the wound.

10. A process according to claim 9, wherein the larvae produce in the wound an alkali wound environment and an elevated temperature favorable to the development of the bacteriophages.

11. A process according to claim 9, wherein the larvae break down wound coverings and bacteria-shielding protective layers by at least one of their secretion and their mouth hooks or teeth.

12. A process for producing a means for wound treatment, in which the secretion of the larvae of flies is applied upon a wound covering, wherein bacteriophages are also applied onto the wound covering.

13. An article for wound treatment, with a wound covering soaked with the secretion of larvae, wherein the wound covering further contains bacteriophages.

14. A process for wound treatment with the secretion of the larvae of flies, wherein bacteriophages are mixed into the secretion.

15. A process according to claim 9, wherein the larvae of flies are of the genus Lucilia.
